(19) United States
(12) Patent Application Publication
(10) Pub. No.: US 2023/0173511 A1
(43) Pub. Date: Jun. 8, 2023

(54) SPRAY DEVICE

(71) Applicant: ADATA TECHNOLOGY CO., LTD., New Taipei City (TW)

(72) Inventors: Chia-Hsiang HSIAO, New Taipei City (TW); CHI-CHIEN LIN, Keelung City (TW); Po-Chun YEN, Taipei City (TW)

(21) Appl. No.: 18/073,589

(22) Filed: Dec. 2, 2022

Related U.S. Application Data
(60) Provisional application No. 63/285,495, filed on Dec. 3, 2021.

(30) Foreign Application Priority Data
Oct. 27, 2022 (TW) 111140814

(51) Int. Cl. B05B 3/02; B05B 3/18

(52) U.S. Cl. CPC B05B 3/021; B05B 3/18; B05B 3/025

(57) ABSTRACT

A spray device includes a controller, a mobile device, a spray tank, a multi-angle spray pipe structure and a nozzle. The controller is arranged on the mobile device and controls the movement of the mobile device. The spray tank includes a liquid reservoir. The multi-angle spray pipe structure is arranged on the spray tank and is communicated with the liquid reservoir. The nozzle is arranged on the multi-angle spray pipe structure. An inlet end plane is formed at an edge of an inlet end of the multi-angle spray pipe structure, an outlet end plane is formed at an edge of an outlet end of the multi-angle spray pipe structure, and the inlet end plane and the outlet end plane intersect to form an included angle.

### FIELD OF THE INVENTION

The present invention relates to a spray device, and more particular to a spray device having a mobile device and a multi-angle spray pipe structure.

### BACKGROUND OF THE INVENTION

Large-scale disinfecting and cleaning operations are often required in places, for example, hospitals, and stations where many people move. Under normal conditions, operators carry handheld sprayers to clean corners in a field domain, which not only consumes manpower, but also cannot guarantee cleaning quality and speed. At present, an automatic disinfection device has been proposed. A spray device is installed on a device (for example, a robot) capable of moving autonomously to perform cleaning and disinfecting operations automatically. However, most nozzles on a disinfection spray vehicle are fixed, and the fixed nozzles cannot fit many special angles or positions to be disinfected, so that cleaning and disinfecting effects are unsatisfactory.

### SUMMARY OF THE INVENTION

The present invention provides a spray device, including a controller, a mobile device, a spray tank, a multi-angle spray pipe structure and a nozzle. The controller is arranged on the mobile device and controls the movement of the mobile device. The spray tank includes a liquid reservoir. The multi-angle spray pipe structure is arranged on the spray tank and is communicated with the liquid reservoir. The nozzle is arranged on the multi-angle spray pipe structure. An inlet end plane is formed at an edge of an inlet end of the multi-angle spray pipe structure, an outlet end plane is formed at an edge of an outlet end of the multi-angle spray pipe structure, and the inlet end plane and the outlet end plane intersect to form an included angle.

In an embodiment of the present invention, the multi-angle spray pipe structure is an elbow pipe.

In an embodiment of the present invention, the spray device further includes an actuating mechanism. The controller is electrically connected to the actuating mechanism to control the multi-angle spray pipe structure to rotate relative to the spray tank.

In an embodiment of the present invention, the spray device further includes a sleeve arranged on the spray tank. The multi-angle spray pipe structure is arranged on the spray tank via the sleeve and is raised or lowered relative to the spray tank via the sleeve.

In an embodiment of the present invention, the multi-angle spray pipe structure includes a first rotary spray pipe and a second rotary spray pipe. The first rotary spray pipe and the second rotary spray pipe each have an inlet end and an outlet end. The inlet end of the first rotary spray pipe is formed in the sleeve. The outlet end of the first rotary spray pipe is communicated with the inlet end of the second rotary spray pipe. The first rotary spray pipe is adapted to rotate relative to the sleeve. The second rotary spray pipe is adapted to rotate relative to the first rotary spray pipe.

In an embodiment of the present invention, an inlet end plane is formed at an edge of the first rotary spray pipe, an outlet end plane is formed at an edge of the outlet end of the first rotary spray pipe, and the inlet end plane of the first rotary spray pipe and the outlet end plane of the first rotary spray pipe intersect to form an included angle. An inlet end plane is formed at an edge of the second rotary spray pipe, an outlet end plane is formed at an edge of the outlet end of the second rotary spray pipe, and the inlet end plane of the second rotary spray pipe and the outlet end plane of the second rotary spray pipe intersect to form an included angle.

In an embodiment of the present invention, the multi-angle spray pipe structure further includes a third rotary spray pipe. The third rotary spray pipe has an inlet end and an outlet end. The inlet end of the third rotary spray pipe is communicated with the outlet end of the second rotary spray pipe. The outlet end of the third rotary spray pipe is communicated with the nozzle. An inlet end plane is formed at an edge of the inlet end of the third rotary spray pipe, an outlet end plane is formed at an edge of the outlet end of the third rotary spray pipe, and the inlet end plane of the third rotary spray pipe and the outlet end plane of the third rotary spray pipe intersect to form an included angle. The third rotary spray pipe is adapted to rotate relative to the second rotary spray pipe.

In an embodiment of the present invention, at least one of the inlet end and the outlet end of the first rotary spray pipe is an inclined end opening, at least one of the inlet end and the outlet end of the second rotary spray pipe is an inclined end opening, and at least one of the inlet end and the outlet end of the third rotary spray pipe is an inclined end opening.

In an embodiment of the present invention, the multi-angle spray pipe structure includes an actuating mechanism. The actuating mechanism includes a first actuating member, a second actuating member and a third actuating member. The controller is electrically connected to the first actuating member, the second actuating member and the third actuating member. The first actuating member is arranged at an abutting joint position of the first rotary spray pipe and the sleeve, the second actuating member is arranged at an abutting joint position of the second rotary spray pipe and the sleeve, and the third actuating member is arranged at an abutting joint position of the third rotary spray pipe and the sleeve, so as to respectively control rotation of the first rotary spray pipe, the second rotary spray pipe and the third rotary spray pipe.

In an embodiment of the present invention, the nozzle includes an outer frame, a plurality of connecting rods and a plurality of scattered pieces. One end of each of the connecting rods is arranged on the outer frame of the nozzle, and the other end of each of the connecting rods is correspondingly connected to a respective one of the scattered pieces.

In an embodiment of the present invention, the nozzle further includes an inner frame. One end of each of the scattered pieces is pivotally arranged on the inner frame of the nozzle, and the connecting rods are adapted to control opening and closing degrees of the scattered pieces.

In an embodiment of the present invention, the outer frame is provided with a plurality of first joints. Each of the scattered pieces is provided with a second joint. Each of the connecting rods is provided with a plurality of installation holes. A spherical top end radially stretches outwards from each of the first joints and the second joint of each of the scattered pieces. The connecting rods are rotatably connected to the spherical top ends via the installation holes.

In an embodiment of the present invention, the liquid reservoir includes a first liquid reservoir and a second liquid reservoir. The multi-angle spray pipe structure is communicated with the first liquid reservoir. The spray device further includes a hose nozzle. The hose nozzle is arranged on the spray tank and is adapted to rotate and move together with the multi-angle spray pipe structure. The hose nozzle is communicated with the second liquid reservoir.

According to the spray device in an embodiment of the present invention, because the spray device has the mobile device and the multi-angle spray pipe structure, the cleaning and disinfecting angle of the multi-angle spray pipe structure can be expanded when the multi-angle spray pipe structure rotates relative to the spray tank on the mobile device as an included angle is formed between the inlet end plane of the multi-angle spray pipe structure and the outlet end plane thereof, and the multi-angle spray pipe structure collocated with movement of the mobile device completes cleaning and disinfecting effects at various angles and in a large scale.

In addition, the multi-angle spray pipe structure is arranged on the spray tank via the sleeve and is raised or lowered relative to the spray tank via the sleeve, so as to complete cleaning and disinfecting operations at different heights.

Furthermore, the nozzle of the spray device in an embodiment of the present invention includes the plurality of scattered pieces and the plurality connecting rods. The opening and closing degrees of the scattered pieces are controlled by virtue of actuation and displacement of the first joint, the second joint and the connecting rods to further adjust the size of the nozzle aperture, so that the spray device can clean and disinfect all positions in an environment at different fluid pressures.

### BRIEF DESCRIPTION OF THE DRAWINGS

The present invention will become more readily apparent to those ordinarily skilled in the art after reviewing the following detailed description and accompanying drawings, in which:

FIG. 1 is a three-dimensional schematic diagram of a spray device of an embodiment of the present invention;

FIG. 2 is a three-dimensional schematic diagram of a multi-angle spray pipe structure of an embodiment of the present invention;

FIG. 3 is a three-dimensional exploded view of a multi-angle spray pipe structure of an embodiment of the present invention;

FIG. 4 is a schematic diagram of an electrical connection between a controller and an actuating mechanism of an embodiment of the present invention;

FIG. 5 is an actuating schematic diagram of a nozzle, a multi-angle spray pipe structure and a sleeve of an embodiment of the present invention; and

FIGS. 6A and 6B are actuating three-dimensional schematic diagrams of a nozzle of an embodiment of the present invention.

### DETAILED DESCRIPTION OF PREFERRED EMBODIMENTS

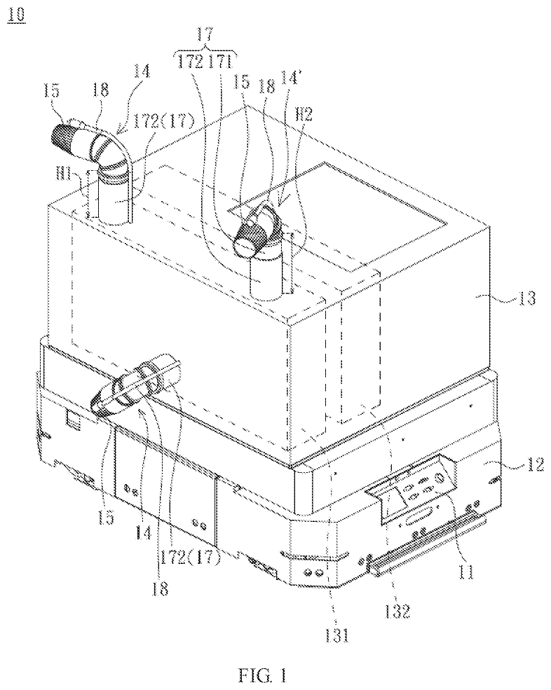

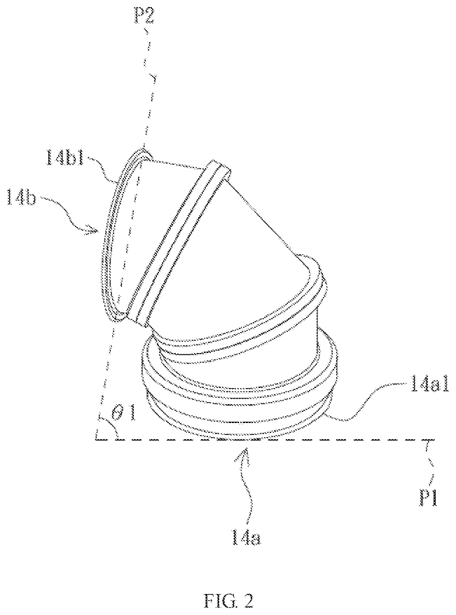

FIG. 1 is a three-dimensional schematic diagram of a spray device in an embodiment of the present invention. The spray device 10 includes a controller 11, a mobile device 12, a spray tank 13, a multi-angle spray pipe structure 14 and a nozzle 15. The controller 11 is arranged on the mobile device 12 and controls movement of the mobile device 12. The spray tank 13 includes a first liquid reservoir 131. The multi-angle spray pipe structure 14 is arranged on the spray tank 13 and is communicated with the first liquid reservoir 131. The nozzle 15 is arranged on the multi-angle spray pipe structure 14. FIG. 2 is a three-dimensional schematic diagram of a multi-angle spray pipe structure of an embodiment of the present invention. Referring to FIG. 2, an inlet end plane P1 is formed at an edge 14a1 of an inlet end 14a of the multi-angle spray pipe structure 14, an outlet end plane P2 is formed at an edge 14b1 of an outlet end 14b of the multi-angle spray pipe structure 14, and the inlet end plane P1 and the outlet end plane P2 intersect to form an included angle θ1, that is, the inlet end plane P1 and the outlet end plane P2 are not parallel to each other. A 360° spray range can be formed when the multi-angle spray pipe structure 14 is arranged on the spray tank 13 and rotates relative to the spray tank 13.

The mobile device 12 can be, for example, an autonomous mobile robot (AMR) or other mobile devices having wheels. The controller 11 arranged on the mobile device 12 can receive an external instruction to control movement of the mobile device 12. Or, the controller 11 can internally include a memory element (not shown), and an operator can set related instructions such as a mobile path of the mobile device 12 in the memory element in advance, and the controller 11 autonomously controls the spray device 10 to perform cleaning and disinfecting operations according to the setting in the memory element based on a set time. The spray tank 13 is arranged on the mobile device 12 and can internally include at least one liquid reservoir such as the first liquid reservoir 131. The first liquid reservoir 131 can be filled with cleaning and disinfecting liquids such as water, alcohol, bleaching water or a detergent. The spray tank 13 can also include other devices, for example, devices capable of mixing or atomizing various liquids such as an atomizer or a stirrer, and the present invention is not limited thereto.

Continued with the above-mentioned description, the spray tank 13 can be provided with at least one multi-angle spray pipe structure 14. In an embodiment shown in FIG. 1, the spray tank 13 is provided with three multi-angle spray pipe structures 14, 14′, wherein two of the multi-angle spray pipe structures are installed on a top surface of the spray tank 13 and the other one is installed on a side surface of the spray tank 13; however, the present invention is not limited thereto. In another embodiment not shown, a multi-angle spray pipe structure 14 can also be installed on the bottom surface of the spray tank 13 when the structure of the mobile device 12 and the spray box 13 allow. The inlet end 14a of the multi-angle spray pipe structure 14 is arranged on the spray tank 13 and can rotate relative to the spray tank 13. Further, the inlet end plane P1₁ and the outlet end plane P2 of the multi-angle spray pipe structure 14 intersect to form an included angle. Therefore, when the inlet end 14a of the multi-angle spray pipe structure 14 rotates relative to the spray tank 13, the outlet end 14b of the multi-angle spray pipe structure 14 swings correspondingly. Because a plurality of multi-angle spray pipe structures 14, 14′ are installed on the spray tank 13 and the outlet ends 14b of the multi-angle spray pipe structures 14, 14′ can swing, a large cleaning and disinfecting range without dead angles can be formed.

In an embodiment of the present invention, the spray device 10 further includes a sleeve 17 arranged on the spray tank 13. Continue to refer to FIG. 1, the multi-angle spray pipe structures 14, 14′ are arranged on the spray tank 13 via the sleeve 17, and the multi-angle spray pipe structures 14, 14′ can be raised or lowered relative to the spray tank 13 via the sleeve 17. In an embodiment, the sleeve 17 has double-layered structures (shown in FIG. 1) capable of rotating relatively and changing a relative position. For example, the sleeve 17 has an inner pipe 171 and an outer pipe 172 having matched spiral track structures (not shown). A relative displacement is formed when the inner pipe 171 or the outer pipe 172 rotates relatively, so as to change the length of the sleeve 17; however, the present invention is not limited thereto. The sleeve 17 can also be, for example, a corrugated pipe with an adjustable length. In the embodiment shown in FIG. 1, the inlet end 14a of the multi-angle spray pipe structures 14, 14′ is connected to the inner pipe 171 of the sleeve 17. When the multi-angle spray pipe structures 14, 14′ rotate relative to the outer pipe 172 of the sleeve 17, the inner pipe 171 of the sleeve 17 is driven to displace relative to the outer pipe 172 to increase the length of the sleeve 17, so as to have various changes in height of the multi-angle spray pipe structure 14 relative to the spray tank 13. As shown in FIG. 1, a height from the multi-angle spray pipe structure 14 installed on the top surface of the spray tank 13 to the spray tank 13 is H1, and this multi-angle spray pipe structure 14 has not yet rotated relative to the outer pipe 172 of the sleeve 17. A height from the other multi-angle spray pipe structure 14′ to the spray tank 13 is H2, and this multi-angle spray pipe structure 14′ has rotated relative to the outer pipe 172 of the sleeve 17, and therefore the multi-angle spray pipe structure 14′ is raised. Due to different rotating degrees of the two multi-angle spray pipe structures 14, 14′ relative to the outer pipe 172 of the sleeve 17, the inner pipe 171 of the sleeve 17 are driven to rotate relative to the outer pipe 172 in different degree, and therefore the length of the sleeve 17 is different. By the relative rotation between the inner pipe 171 and the outer pipe 172 of the sleeve 17, the multi-angle spray pipe structure 14 can be raised or lowered relative to the spray tank 13, and therefore the spray operations for the positions at different heights can be performed.

Please continue to refer to FIG. 2. The inlet end plane P1 and the outlet end plane P2 of the multi-angle spray pipe structure 14 form an included angle θ1, and a range of the included angle θ1₁ can be, for example, 1° to 135°. As shown in FIG. 2, the multi-angle spray pipe structure 14 can be, for example, an elbow pipe; and accordingly the included angle θ1 formed between the inlet end plane P1₁ and the outlet end plane P2 can be, for example, an included angle approximates to 90°.

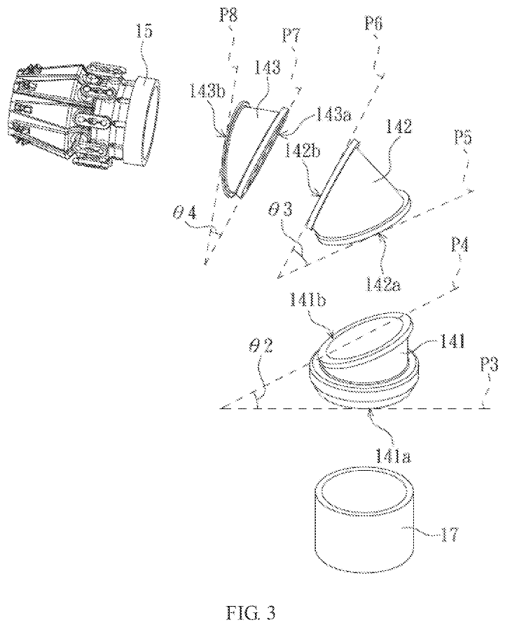

Referring to FIG. 3, FIG. 3 is a three-dimensional exploded view of a multi-angle spray pipe structure of an embodiment of the present invention. The multi-angle spray pipe structure 14 can include a first rotary spray pipe 141, a second rotary spray pipe 142 and a third rotary spray pipe 143. The first rotary spray pipe 141, the second rotary spray pipe 142 and the third rotary spray pipe 143 respectively include the inlet ends 141a, 142a, 143a and the outlet ends 141b, 142b, 143b. The inlet end planes P3, P5, P7 are respectively formed at the edges of the inlet ends 141a, 142a, 143a, and the outlet end planes P4, P6, P8 are respectively formed at the edges of the outlet ends 141b, 142b, 143b. The inlet end plane P3 and the outlet end plane P4 of the first rotary spray pipe 141 intersect to form an included angle θ2; the inlet end plane P5 and the outlet end plane P6 of the second rotary spray pipe 142 intersect to form an included angle θ3; and the inlet end plane P7 and the outlet end plane P8 of the third rotary spray pipe 143 intersect to form an included angle θ4. The included angles θ2, θ3 and θ4 can be ranged, for example, from 1° to 45°. In the embodiment where the multi-angle spray pipe structure 14 includes the first rotary spray pipe 141, the second rotary spray pipe 142 and the third rotary spray pipe 143, the sum of the included angles θ2₂, θ3 and θ4 is equal to the included angle θ1 formed between the inlet end plane P1 and the outlet end plane P2 of the multi-angle spray pipe structure 14; the inlet end plane P3 of the first rotary spray pipe 141 is the inlet end plane P1 of the multi-angle spray pipe structure 14; and the outlet end plane P8 of the third rotary spray pipe 143 is the outlet end plane P2 of the multi-angle spray pipe structure 14.

Continue the description. The inlet end 141a of the first rotary spray pipe 141 is arranged on the sleeve 17, the outlet end 141b of the first rotary spray pipe 141 is communicated with the inlet end 142a of the second rotary spray pipe 142, the outlet end 142b of the second rotary spray pipe 142 is communicated with the inlet end 143a of the third rotary spray pipe 143, and the outlet end 143b of the third rotary spray pipe 143 is communicated with the nozzle 15; however, the present invention is not limited thereto. The arrangements of the second rotary spray pipe 142 and the second rotary spray pipe 143 can be selected based on a demand of a user.

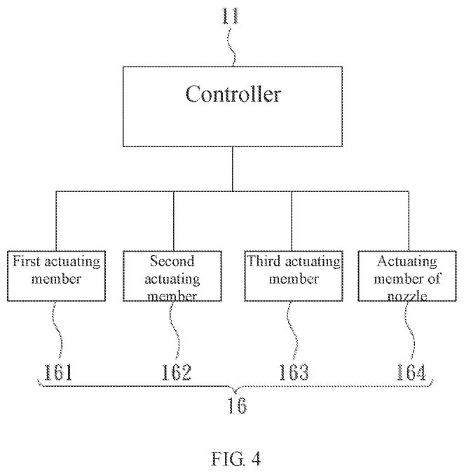

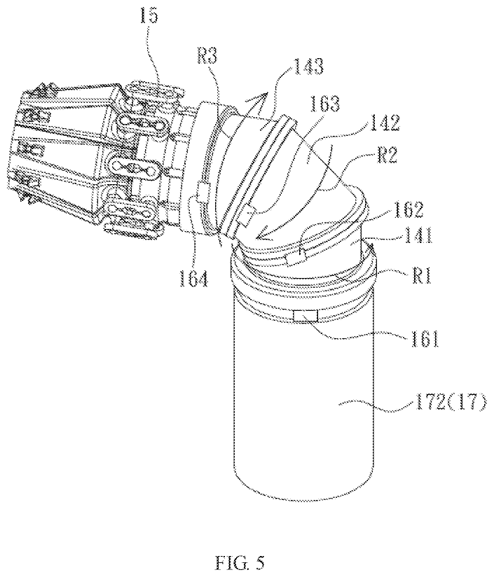

Refer to FIGS. 4 and 5. FIG. 4 is a schematic diagram of an electrical connection between a controller and an actuating mechanism of an embodiment of the present invention. FIG. 5 is an actuating schematic diagram of a nozzle, a multi-angle spray pipe structure and a sleeve of an embodiment of the present invention. In an embodiment, the spray device 10 further includes an actuating mechanism 16, and the actuating mechanism 16 includes a first actuating member 161, a second actuating member 162 and a third actuating member 163. As shown in FIG. 4, the controller 11 is electrically connected to the first actuating member 161, the second actuating member 162 and the third actuating member 163. The first actuating member 161 is arranged between the first rotary spray pipe 141 and the sleeve 17 and is configured to control the first rotary spray pipe 141 to rotate relative to the outer pipe 172 of the sleeve 17. Further, the rotation of the first rotary spray pipe 141 can drive the matched spiral track structures between the double-layered structures of the sleeve 17 to move. Therefore, when the first rotary spray pipe 141 rotates relative to the sleeve 17, besides changing an injection direction of the outlet end 141b of the first rotary spray pipe 141, the first rotary spray pipe 141 can also have a raised and lowered effect relative to the spray tank 13. The second actuating member 162 is arranged at a junction between the first rotary spray pipe 141 and the second rotary spray pipe 142 and is configured to control the second rotary spray pipe 142 to rotate relative to the first rotary spray pipe 141. The third actuating member 163 is arranged at a junction between the second rotary spray pipe 142 and the third rotary spray pipe 143 and is configured to control the third rotary spray pipe 143 to rotate relative to the second rotary spray pipe 142. By configuring the controller 11 to control the first actuating member 161, the second actuating member 162 and the third actuating member 163, the relative angles between the first rotary spray pipe 141, the second rotary spray pipe 142 and the third rotary spray pipe 143 are changed, so that the directions of the multi-angle spray pipe structure 14 spraying a cleaning liquid can be adjusted in three rotatable axial directions, and therefore the cleaning and disinfecting operation of the multi-angle spray pipe structure 14 is better. In the embodiment, the first actuating member 161, the second actuating member 162 and the third actuating member 163 can be, for example, motors (not shown) electrically connected to the controller 11 to automatically control the direction of the multi-angle spray pipe structure 14 via the controller 11; however, the present invention is not limited thereto. In an embodiment, the first actuating member 161, the second actuating member 162 and the third actuating member 163 can be structures such as worms or gear rings, and therefore the relative positions and directions of the elements such as the sleeve 17, the first rotary spray pipe 141, the second rotary spray pipe 142 and the third rotary spray pipe 143 can be manually adjusted by a user.

In the embodiment shown in FIG. 5, the inlet end 141a of the first rotary spray pipe 141 is formed in the sleeve 17 and can, for example, rotate along a rotating direction R1 relative to the outer pipe 172 of the sleeve 17, wherein the rotating direction R1 can be a direction rotting the axis of the sleeve 17. The outlet end 141b of the first rotary spray pipe 141 and the inlet end 142a of the second rotary spray pipe 142 are inclined end openings in abutting joint. The second rotary spray pipe 142 rotates along a rotating direction R2 relative to the first rotary spray pipe 141, so as to form a rotating direction in another dimension. The inlet end 143a of the third rotary spray pipe 143 and the outlet end 142b of the second rotary spray pipe 142 are inclined end openings in abutting joint. The third rotary spray pipe 143 rotates along a rotating direction R3 relative to the second rotary spray pipe 142. Further, the outlet end 143b of the third rotary spray pipe 143 is connected to a nozzle 15, and the nozzle 15 connected to a tail end of the multi-angle spray pipe structure 14 can have three rotating directions due to the relative rotation of the first rotary spray pipe 141, the second rotary spray pipe 142 and the third rotary spray pipe 143, so that the nozzle 15 has flexible angle and direction while cleaning and disinfecting. In an embodiment, the aforementioned inclined end opening refers to that the length from the inlet end to the outlet end of the rotary spray pipe changes along a circumferential direction at the edge of the inlet end, so that the inlet end plane and the outlet plane of the rotary spray pipe are not parallel to each other and are incline relatively to form an included angle. In this embodiment, the relative inclination between the inlet end plant and the outlet end plane is achieved by the inclined end opening; however, the present invention is not limited thereto. The rotary spray pipe can also include a part of flexible pipe body, so that the outlet end of the rotary spray pipe can incline relative to the inlet end.

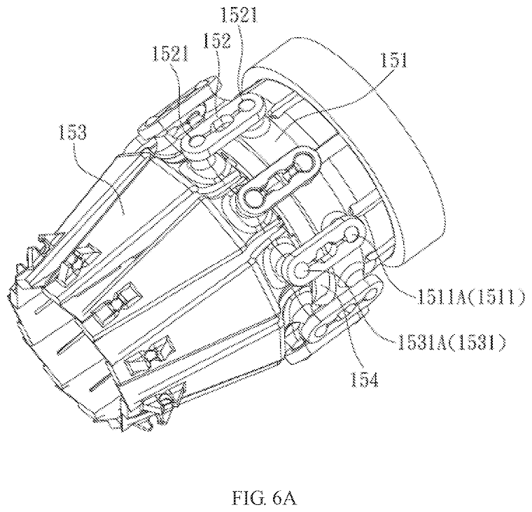

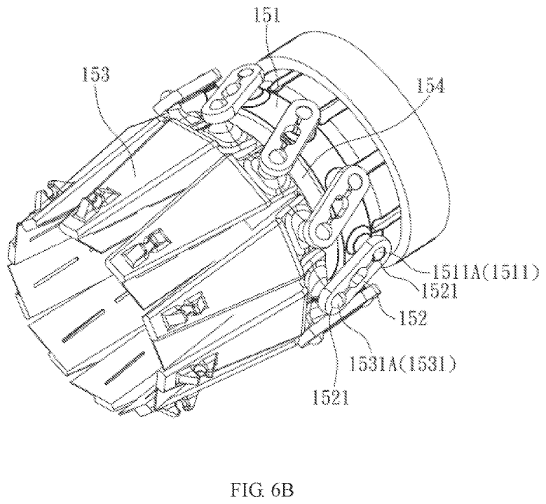

Referring to FIGS. 6A and 6B, FIGS. 6A and 6B are three-dimensional schematic diagrams of an actuating of a nozzle of an embodiment of the present invention. Specifically, FIG. 6A is a schematic diagram of a condensed nozzle aperture. As shown in the figure, the nozzle 15 includes an outer frame 151, a plurality of scattered pieces 153, a plurality of connecting rods 152 and an inner frame 154. The outer frame 151 includes a plurality of first joints 1511 distributed in a circumferential direction, and a spherical top end 1511A radially stretches outwards from each of the first joints 1511. The scattered pieces 153 are pivotally arranged in the inner frame 154. Each of the scattered pieces 153 is correspondingly connected to a connecting rod 152. The end of the scattered piece 153 connected to the connecting rod 152 is provided with a second joint 1531. A spherical top end 1531A radially stretches outwards from the respective second joint 1531. The other ends of the plurality of scattered pieces 153 not connected to the connecting rods 152 form the outlet end of the nozzle 15. The connecting rod 152 is provided with a plurality of installation holes 1521, and the shapes of the installation holes 1521 can be substantially circular to correspond to the spherical top end 1511A of the first joint 1511 and the spherical top end 1531A of the second joint 1531. The connecting rod 152 is rotatably connected to the spherical top end 1511A of the first joint 1511 and the spherical top end 1531A of the second joint 1531 via the installation holes 1521. The outer frame 151 and the inner frame 154 have not yet rotate relatively in the nozzle 15 shown in FIG. 6A, and therefore the length direction of the connecting rod 152 and the spray direction of the nozzle 15 are parallel to each other, so that the distances between the spherical top end 1511A of the first joint 1511 and the spherical top end 1531A of the second joint 1531 are maximized in the spray direction. Therefore, the ends of the plurality of scattered pieces 153 as the outlet end of the nozzle 15 are gathered to reduce the aperture and the sectional area of the nozzle 15. Referring to FIG. 6B, FIG. 6B is a schematic diagram of an expanded nozzle aperture. In a case where the aperture of the nozzle 15 is to be increased, the nozzle actuating member 164 (shown in FIG. 5) connected to the nozzle 15 drives the outer frame 151 to rotate relative to the inner frame 154. When the outer frame 151 and the inner frame 154 have a relative displacement along the circumferential direction, the first joint 1511 is driven to move in the circumferential direction relative to the second joint 1531. In an embodiment, the connecting rod 152 can be a member having a fixed structure, that is, the distance between the installation holes 1521 in the connecting rod 152 is fixed. Therefore, the connecting rod 152 rotates at an angle relative to the spray direction of the nozzle 15 when the first joint 1511 moves in the circumferential direction relative to the second joint 1531, so that the spherical top end 1531A of the second joint 1531 approaches the outer frame 151 and drives the scattered pieces 153 pivotably connected to the inner frame 154 to be unfolded outwards to form a large nozzle aperture. According to the nozzle 15 in an embodiment of the present invention, by configuring the nozzle actuating member 164 to drive the first joint 1511 and the second joint 1531 to have a specific relative displacement in the rotating direction, the opening and closing degrees of the plurality of scattered pieces 153 are controlled so as to adjust the aperture or the sectional area of the nozzle 15, thereby changing the fluid pressure when the cleaning and disinfecting liquid is sprayed. In this embodiment, the nozzle 15 further drives the displacements of the connecting rod 152 and the second joint 1531 by rotating the first joint 1511 so as to control the opening and closing of the scattered pieces 153; however, the present invention is not limited thereto. The connecting rod can also be a connecting rod with flexibility. The connecting rod is elongated or shortened to regulate and control the gathering degrees of the plurality of scattered pieces at the outlet end of the nozzle.

Referring to FIG. 1 again, the spray device 10 in an embodiment of the present invention can further include one or more hose nozzles 18. The hose nozzle 18 is arranged on the spray tank 13 and is adapted to rotate and move together with the multi-angle spray pipe structure 14. The spray tank 13 can internally include a plurality of liquid reservoirs, such as a first liquid reservoir 131 and a second liquid reservoir 132. The multi-angle spray pipe structure 14 can be, for example, communicated with the first liquid reservoir 131. The hose nozzle 18 can be, for example, communicated with the second liquid reservoir 132. Thus, the spray device 10 can perform cleaning and disinfection with two or more liquids. In another embodiment, a plurality of multi-angle spray pipe structures 14 are installed on the spray tank 13, and different multi-angle spray pipe structures 14 are communicated with different liquid reservoirs. In an embodiment, the spray tank 13 can be internally provided with a plurality of internal communicating pipelines (not shown) to communicate the hose nozzles 18 and the multi-angle spray pipe structures 14 to different liquid reservoirs.

According to the spray device in an embodiment of the present invention, the mobile device and the multi-angle spray pipe structure are integrated, and an included angle is formed between the inlet end plane of the multi-angle spray pipe structure and the outlet end plane thereof. Thus, the cleaning and disinfecting angle of the multi-angle spray pipe structure can be expanded when the multi-angle spray pipe structure rotates relative to the spray tank on the mobile device, and the multi-angle spray pipe structure collocated with movement of the mobile device completes cleaning and disinfecting effects at various angles and in a large scale. Furthermore, the multi-angle spray pipe structure can include a plurality of rotary spray pipes. The plurality of rotary spray pipes can rotate relatively, and the multi-angle spray pipe structure is arranged on the sleeve and can be raised or lowered relative to the spray tank, so that the multi-angle spray pipe structure can have a plurality of rotating directions and a changeable injection height, thereby increasing the flexibility of the cleaning and disinfecting operation and improving the defect that the cleaning and disinfecting effect is poor as the fixed nozzle on the conventional cleaning and disinfecting device cannot cooperate with to-be-disinfected special angles or positions.

In addition, the nozzle of the spray device in an embodiment of the present invention includes the plurality of scattered pieces and the plurality connecting rods. The opening and closing degrees of the scattered pieces are controlled by virtue of actuation and displacement of the first joint, the second joint and the connecting rods to further adjust the size of the nozzle aperture, so that the spray device can clean and disinfect all positions in an environment at different fluid pressures.

Finally, it should be noted that the above embodiments are merely intended to illustrate rather than to limit the technical solution of the present invention. Although the present invention has been described in detail with reference to the preferred embodiments, those of ordinary skill in the art should understand that the technical solution of the present invention may be modified or equivalently substituted without departing from the objective and scope of the technical solution of the present invention, which should be covered in the scope of claims of the present invention.

What is claimed is:

1. A spray device, comprising:
a controller;
a mobile device, arranged on the mobile device and controlling a movement of the mobile device;
a spray tank, comprising at least one liquid reservoir;
a multi-angle spray pipe structure, arranged on the spray tank and communicated with the at least one liquid reservoir; and
a nozzle, arranged on the multi-angle spray pipe structure,
wherein an inlet end plane is formed at an edge of an inlet end of the multi-angle spray pipe structure, an outlet end plane is formed at an edge of an outlet end of the multi-angle spray pipe structure, and the inlet end plane and the outlet end plane intersect to form an included angle.

2. The spray device according to claim 1, wherein the multi-angle spray pipe structure is an elbow pipe.

3. The spray device according to claim 1, further comprising an actuating mechanism, wherein the controller is electrically connected to the actuating mechanism to control the multi-angle spray pipe structure to rotate relative to the spray tank.

4. The spray device according to claim 1, further comprising a sleeve arranged on the spray tank, wherein the multi-angle spray pipe structure is arranged on the spray tank via the sleeve and is raised or lowered relative to the spray tank via the sleeve.

5. The spray device according to claim 4, wherein the multi-angle spray pipe structure comprises a first rotary spray pipe and a second rotary spray pipe, the first rotary spray pipe and the second rotary spray pipe each have an inlet end and an outlet end, the inlet end of the first rotary spray pipe is formed in the sleeve, the outlet end of the first rotary spray pipe is communicated with the inlet end of the second rotary spray pipe, the first rotary spray pipe is adapted to rotate relative to the sleeve, and the second rotary spray pipe is adapted to rotate relative to the first rotary spray pipe.

6. The spray device according to claim 5, wherein an inlet end plane is formed at an edge of the first rotary spray pipe, an outlet end plane is formed at an edge of the outlet end of the first rotary spray pipe, and the inlet end plane of the first rotary spray pipe and the outlet end plane of the first rotary spray pipe intersect to form an included angle; an inlet end plane is formed at an edge of the second rotary spray pipe, an outlet end plane is formed at an edge of the outlet end of the second rotary spray pipe, and the inlet end plane of the second rotary spray pipe and the outlet end plane of the second rotary spray pipe intersect to form an included angle.

7. The spray device according to claim 6, wherein the multi-angle spray pipe structure further comprises a third rotary spray pipe, the third rotary spray pipe has an inlet end and an outlet end, the inlet end of the third rotary spray pipe is communicated with the outlet end of the second rotary spray pipe, the outlet end of the third rotary spray pipe is communicated with the nozzle, an inlet end plane is formed at an edge of the inlet end of the third rotary spray pipe, an outlet end plane is formed at an edge of the outlet end of the third rotary spray pipe, the inlet end plane of the third rotary spray pipe and the outlet end plane of the third rotary spray pipe intersect to form an included angle, and the third rotary spray pipe is adapted to rotate relative to the second rotary spray pipe.

8. The spray device according to claim 7, wherein at least one of the inlet end and the outlet end of the first rotary spray pipe is an inclined end opening, at least one of the inlet end and the outlet end of the second rotary spray pipe is an inclined end opening, and at least one of the inlet end and the outlet end of the third rotary spray pipe is an inclined end opening.

9. The spray device according to claim 7, further comprising an actuating mechanism, wherein the actuating mechanism comprises a first actuating member, a second actuating member and a third actuating member, the controller is electrically connected to the first actuating member, the second actuating member and the third actuating member, the first actuating member is arranged at an abutting joint position of the first rotary spray pipe and the sleeve, the second actuating member is arranged at an abutting joint position of the second rotary spray pipe and the sleeve, and the third actuating member is arranged at an abutting joint position of the third rotary spray pipe and the sleeve, so as to respectively control rotation of the first rotary spray pipe, the second rotary spray pipe and the third rotary spray pipe.

10. The spray device according to claim 1, wherein the nozzle comprises an outer frame, a plurality of connecting rods and a plurality of scattered pieces, one end of each of the connecting rods is arranged on the outer frame of the nozzle, and the other end of each of the connecting rods is correspondingly connected to a respective one of the scattered pieces.

11. The spray device according to claim 10, wherein the nozzle further comprises an inner frame, one end of each of the scattered pieces is pivotally arranged on the inner frame of the nozzle, and the connecting rods are adapted to control opening and closing degrees of the scattered pieces.

12. The spray device according to claim 11, wherein the outer frame is provided with a plurality of first joints, each of the scattered pieces is provided with a second joint, each of the connecting rods is provided with a plurality of installation holes, a spherical top end radially stretches outwards from each of the first joints and the second joint of each of the scattered pieces, and the connecting rods are rotatably connected to the spherical top ends via the installation holes.

13. The spray device according to claim 1, wherein the at least one liquid reservoir comprises a first liquid reservoir and a second liquid reservoir, the multi-angle spray pipe structure is communicated with the first liquid reservoir, the spray device further comprises a hose nozzle, the hose nozzle is arranged on the spray tank and is adapted to rotate and move together with the multi-angle spray pipe structure, and the hose nozzle is communicated with the second liquid reservoir.
